(19) United States
(12) Patent Application Publication
(10) Pub. No.: US 2023/0177228 A1
(43) Pub. Date: Jun. 8, 2023

(54) SYSTEMS AND METHODS FOR ASSESSING ERGONOMIC RISK

(71) Applicant: Dassault Systemes Americas Corp., Waltham, MA (US)

(72) Inventors: Quentin Bourret, Montreal (CA); Julie Charland, Montreal (CA); Daniel Imbeau, Montreal (CA); David Brouillette, Saint-Lambert (CA); Jean-Baptist Djire, Montreal (CA)

(21) Appl. No.: 18/063,338

(22) Filed: Dec. 8, 2022

Related U.S. Application Data
(60) Provisional application No. 63/287,251, filed on Dec. 8, 2021.

(51) Int. Cl. G06F 30/17; G16H 50/30

(52) U.S. Cl. CPC G06F 30/17; G16H 50/30

(57) ABSTRACT

Embodiments assess ergonomic risk in environments, such as factories and workstations. One such embodiment begins by receiving process planning data for an operator performing a task. In turn, the received process planning data is used to generate a posture for the operator to perform the task in a certain real-world environment. The generated posture is processed using a hierarchical decision tree to determine ergonomic risk of the posture in the certain real-world environment. Output includes an indication of the determined ergonomic risk.

### RELATED APPLICATION

This application claims the benefit of U.S. Provisional Application No. 63/287,251, filed on Dec. 8, 2021.

The entire teachings of the above application are incorporated herein by reference.

### BACKGROUND

A number of existing product and simulation systems are offered on the market for the design and simulation of objects, e.g., humans, parts, and assemblies of parts, amongst other examples. Such systems typically employ computer aided design (CAD) and/or computer aided engineering (CAE) programs. These systems allow a user to construct, manipulate, and simulate complex three-dimensional (3D) models of objects or assemblies of objects. These CAD and CAE systems, thus, provide a representation of modeled objects using edges, lines, faces, polygons, or closed volumes. Lines, edges, faces, polygons, and closed volumes may be represented in various manners, e.g., non-uniform rational basis-splines (NURBS).

CAD systems manage parts or assemblies of parts of modeled objects, which are mainly specifications of geometry. In particular, CAD files contain specifications, from which geometry is generated. From geometry, a representation is generated. Specifications, geometries, and representations may be stored in a single CAD file or multiple CAD files. CAD systems include graphic tools for representing the modeled objects to designers; these tools are dedicated to the display of complex objects. For example, an assembly may contain thousands of parts. A CAD system can be used to manage models of objects, which are stored in electronic files.

CAD and CAE systems use of a variety of CAD and CAE models to represent objects. These models may be programmed in such a way that the model has the properties (e.g., physical, material, or other physics based) of the underlying real-world object or objects that the model represents. Moreover, CAD/CAE models may be used to perform simulations of the real-word objects/environments that the models represent.

### SUMMARY

Simulating an operator, e.g., a human represented by a digital human model (DHM), in an environment is a common simulation task implemented and performed by CAD and CAE systems. Here, an operator refers to an entity which can observe and act upon an environment, e.g., a human, an animal, or a robot, amongst other examples. Computer-based operator simulations can be used to automatically predict behavior of an operator in an environment when performing a task with one or more target objects. To illustrate one such example, these simulations can determine position and orientation of a human when assembling a car in a factory. The results of the simulations can, in turn, be used to improve the physical environment. For example, simulation results may indicate that ergonomics or manufacturing efficiency can be improved by relocating objects in the environment.

Advantageously, DHMs and existing simulation technologies offer a unique possibility to evaluate risks for a worker, e.g., before a production line is built or for purposes of improving an existing real-world workstation. However, in order to evaluate risks using current DHM software, ergonomics knowledge is required to interpret the results of the ergonomic methods. Further, existing DHM software solutions are also complex to use for engineers while they are designing workstations, especially when posturing the manikins [1] (bracketed numbers in this document refer to the enumerated list of references hereinbelow).

Embodiments solve these problems and provide improved functionality for evaluating risks, e.g., assessing ergonomic risks for workers.

One such embodiment is directed to a computer-implemented method of assessing ergonomic risk. The method begins by receiving process planning data for an operator performing a task. Next, the received process planning data is used to generate a posture for the operator to perform the task, e.g., in a certain real-world environment. In turn, the generated posture is processed, i.e., analyzed, using a hierarchical decision tree to determine ergonomic risk of the posture in the certain real-world environment. The method then outputs an indication of the determined ergonomic risk. The output indication is displayed, audibly rendered and/or provided through tactile means for user correction, warning, and the like for non-limiting example.

According to an embodiment, the process planning data includes at least one of physical characteristics of a workstation in the certain real-world environment at which the task is performed, physical characteristics of the operator, and characteristics of the task. Further, an embodiment receives the process planning data by receiving a measurement from a sensor in the certain real-world environment in which the task is performed.

In an embodiment, processing the generated posture using the hierarchical decision tree to determine ergonomic risk of the posture comprises evaluating existence of multiple risk types of the posture in a hierarchical order of the multiple risk types. In such an embodiment, upon determining a given risk type of the multiple risk types exists, the evaluation is stopped. Further, the embodiment outputs an indication of the determined ergonomic risk that includes an indication of the given risk type. In other words, such an embodiment indicates which of the determined risk types was identified.

According to an embodiment, the hierarchical order of the multiple risk types, in order from first evaluated to last evaluated, includes: object weight risk type, hand position risk type, joint load risk type, and body joint angle risk type.

An embodiment evaluates the existence of the object weight risk type by comparing weight of an object grasped by the operator performing the task to a threshold and concludes the object weight risk type exists if the weight of the object exceeds the threshold. According to an embodiment, a value of the threshold changes based upon the object being grasped with one hand or two hands. Another embodiment evaluates existence of the joint load risk type by: (i) determining at least one of back joint load, shoulder joint load, and elbow joint load of the operator in the generated posture, (ii) comparing the determined at least one of back joint load, shoulder joint load, and elbow joint load to a threshold, and (iii) determining the joint load risk type exists if the determined at least one of back joint load, shoulder joint load, and elbow joint load exceeds the threshold. Further, in an embodiment, evaluating existence of the joint angle risk type includes comparing at least one of shoulder angle, trunk angle, neck angle, wrist angle, and forearm angle of the operator in the generated posture to respective thresholds. Such an embodiment concludes the joint angle risk type exists if at least one of the angles (shoulder angle, trunk angle, neck angle, wrist angle, and forearm angle) exceeds a respective threshold.

An embodiment outputs the indication of the determined ergonomic risk to at least one user. According to an embodiment, the output indication of the determined ergonomic risk includes at least one of: a risk type, a risk location, a risk level, and a suggestion to lower risk. In an example embodiment where the indication of the determined ergonomic risk includes a suggestion to lower risk, the suggestion is determined by searching a mapping between risk types, risk locations, and suggestions. In such an embodiment the determined suggestion is mapped to a given risk type and a given risk location of the determined ergonomic risk. Embodiments can also implement the suggestions in real-world environments.

Another embodiment is directed to a system for assessing ergonomic risk. According to an embodiment, the system includes a processor and a memory with computer code instructions stored thereon. In such an embodiment, the processor and the memory, with the computer code instructions, are configured to cause the system to implement any embodiments or combination of embodiments described herein.

Yet another embodiment is directed to a cloud computing implementation for assessing ergonomic risk. Such an embodiment is directed to a computer program product executed by a server in communication across a network with one or more client. The computer program product comprises program instructions which, when executed by a processor, causes the processor to implement any embodiments or combination of embodiments described herein.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a flowchart of a method for assessing ergonomic risk according to an embodiment.

FIG. 2 illustrates an example interface for providing process planning data in an embodiment.

FIG. 3 illustrates a hierarchical processing workflow that may be employed in embodiments.

FIG. 4 is a flowchart of a method for assessing object weight risk according to an embodiment.

FIG. 5 is a flowchart of a method for assessing hand position risk according to an embodiment.

FIG. 6 is a flowchart of a method for assessing joint load risk according to an embodiment.

FIG. 7 is a flowchart of a method for assessing joint angle risk according to an embodiment.

FIG. 8 depicts example output that may be provided by embodiments.

FIG. 9 is an example output visualization that may be provided by embodiments.

FIG. 10 is a table of ergonomic risk mitigation suggestion mappings that may be used in embodiments.

FIG. 11 is a simplified diagram of a computer system for assessing ergonomic risk according to an embodiment.

FIG. 12 is a simplified diagram of a computer network environment in which an embodiment of the present invention may be implemented.

### DETAILED DESCRIPTION

A description of example embodiments follows.

Occupational ergonomics have a significant impact in the manufacturing world, from Musculoskeletal Disorders (MSD) to product quality issues. As such, assessing ergonomics through the use of models, e.g., digital human models (DHMs), is an important task for organizations, e.g., manufacturers.

Stephens and Jones [1] explain that “DHM[s] have increased the ability to determine risk and acceptability of design very early in the product development cycle.” The main advantages of DHMs are the amount of biomechanical and anthropometrical data that is available. DHMs allow users to compare different scenarios, e.g., manufacturing scenarios, in a measurable way. Several applications, such as Santos [2], Jack (Siemens) [3], DELMIA Ergonomics (Dassault Systemes) [4], allow the use of DHMs in a 3D manufacturing context.

However, Stephens [1] highlights that one of the challenges of existing DHM applications lies in their low efficiency in the process of placing a manikin (i.e., DHM) in a 3D environment. This difficulty arises because users have to posture the manikin manually by moving each joint separately. This process is very time consuming. Recently, Jack [5] and IPS IMMA [6] have published some work that shows posture automation inside their software. However, the posture generation is not yet fully automatic as there is still a need to place a manikin close to the object, but it is a step forward in shortening the manikin posturing duration.

When a posture is generated, a number of methods are available to evaluate the simulated environment, such as RULA (Rapid Upper Limb Assessment) [7], REBA Rapid Entire Body Assessment [8], and revised NIOSH lifting equation [9], amongst others. However, Chaffin [10] showed in a survey that less than 10% of engineers can show at least one complete course in human factor and ergonomics in their background. This means that even if a posture can be easily generated for a DHM, users (e.g., manufacturing engineers, production line managers, other workplace personnel) likely do not know which method to use to assess the simulated environment, nor how to interpret a method’s results. As such, existing methods do not allow users to evaluate ergonomics, e.g., for a human performing a task, and do not provide actionable advice to improve ergonomics responsive to evaluations.

Embodiments, which may be referred to herein as Ergo4All™, solve these problems. Embodiments can be integrated inside an ergonomic simulation application, such as Ergonomic Workplace Design (EWD) [4]. Such applications allow users to test real-world environments and tasks performed in those environments. For example, EWD can be used to evaluate a manufacturing production line, such as a vehicle assembly line, and tasks performed on the production line, such as welding a vehicle chassis, in three-dimensions. Evaluated environments, e.g., production lines, can exist in the real-world or the simulation applications can be used to test and evaluate proposed environment designs before construction.

Embodiments can reuse process planning data from existing ergonomic simulation applications, such as, EWD. Embodiments can also utilize a three-dimensional (3D) representation of worker tasks, such as those developed using EWD. Embodiments can, in turn, use the work place process planning data in a manikin posturing engine, such as SPE™, to automatically generate a manikin posture, e.g., fully automatically, in a 3D environment as given by the workplace data [11-13]. In turn, embodiments, e.g., Ergo4All™, analyze the work situation, that is, a mannikin performing an action in a 3D environment. Embodiments identify the MSD risk level, as well as the risk type and body area at risk. Embodiments also provide suggestions to improve the environment, e.g., workstation, design to lower the MSD-related risk for the worker. Embodiments provide manufacturing engineers with ergonomics knowledge that they often lack and allows users to perform rapid assessments while designing workstations early in a design process.

Embodiments, e.g., Ergo4All™, provide such functionality by employing a hierarchical decision tree to assess the risk of developing MSDs in a 3D simulation of a work situation (e.g., workstation along a production line). Embodiments perform a MSD risk evaluation and provide suggestions to users on ways to improve designs, e.g., workplace designs.

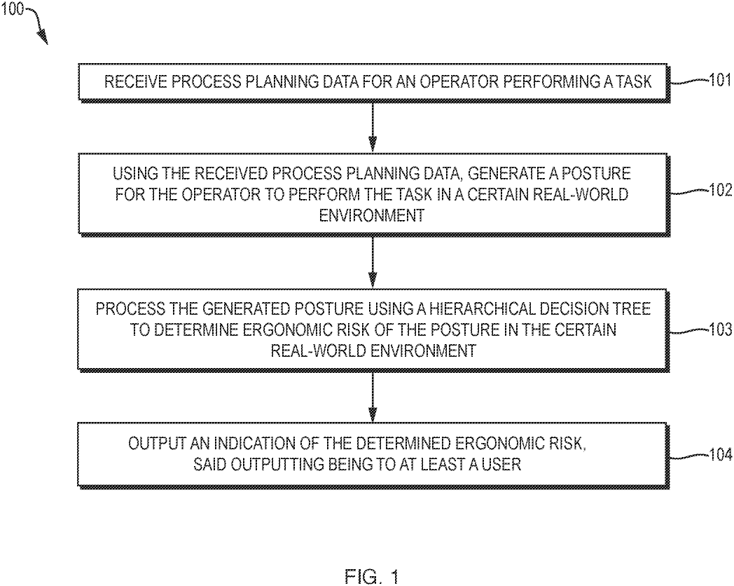

FIG. 1 is a flowchart of one such example embodiment. The method 100 begins at step 101 by receiving process planning data for an operator performing a task. Next, the received process planning data is used at step 102 to generate a posture for the operator to perform the task in a certain real-world environment. In turn, the generated posture is processed, i.e., analyzed, at step 103 using a hierarchical decision tree to determine ergonomic risk of the posture in the certain real-world environment. The method 100 then outputs 104, e.g., to a user, an indication (visual, audible, tactile, or any combination) of the determined ergonomic risk.

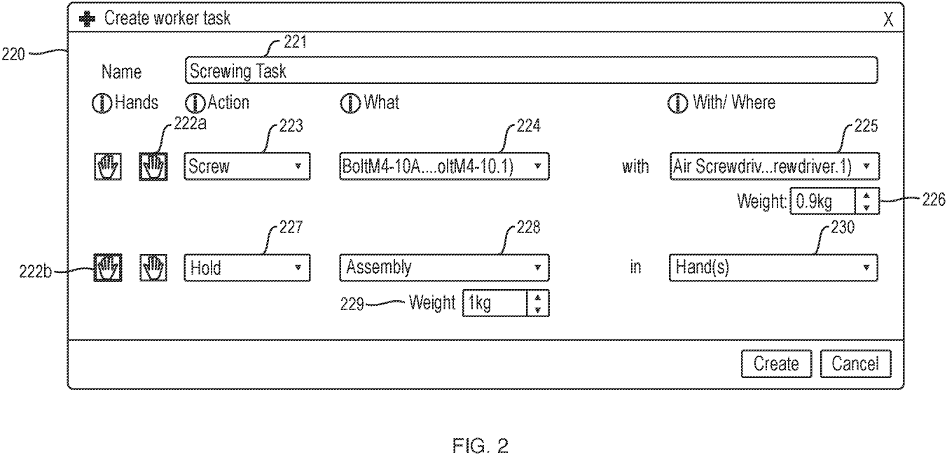

The process planning data received at step 101 can include any information regarding the task, environment, and scenario being analyzed. For instance, the data can include physical characteristics, e.g., dimensions, of a workstation in the real-world environment at which the task is performed. Likewise, the process planning data can include physical characteristics, e.g., height, weight, body dimensions, etc., of the operator. Moreover, the process planning data can include characteristics, e.g., a definition of the task. For instance, such data may indicate that the task is tightening a bolt on a beam with a socket wrench. Further, because the method 100 is computer implemented, the process planning data may be received at step 101 from any memory or data storage that can be communicatively coupled to a computing device implementing the method 100. Further, in an embodiment of the method 100 that is being used to evaluate a real-world environment, the process planning data is received at step 101 by receiving a measurement from a sensor in the certain real-world environment in which the task is performed. Further still, the process planning data may be received from a user or responsive to user input. In an embodiment, the process planning data is provided by a user utilizing the interface 220 described hereinbelow in relation to FIG. 2.

According to an embodiment, the posture generated at step 102 is an indication in three-dimensional space of whole-body position and orientation of the operator to perform the task. In an embodiment, each body segment is defined by its joint angles and segment lengths. An embodiment of the method 100 utilizes existing posture generation software at step 102 to generate the posture. Such existing software is configured to generate posture of a model, e.g., a DHM, representing an operator. Amongst other examples, embodiments may utilize the Smart Posturing Engine™ [11, 12, 13, 24] developed by the applicant-assignee to generate the posture.

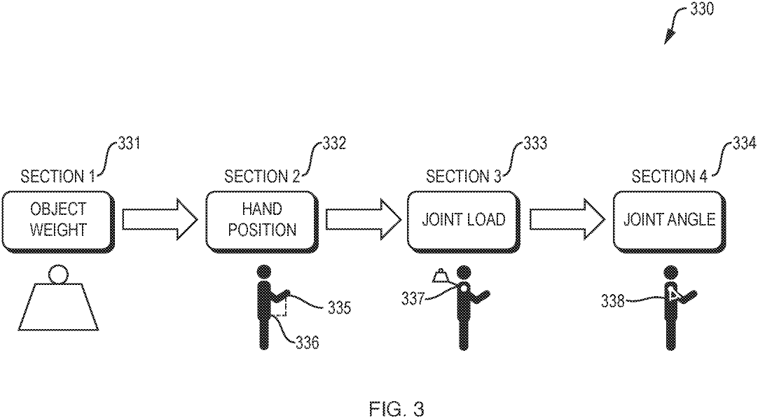

The processing 103, according to an embodiment of the method 100, implements the methods 330, 440, 550, 660, and/or 770 described hereinbelow in relation to FIGS. 3-7, respectively. In an embodiment, the processing 103 implements the methods 440, 550, 660, and 770 in that order, but stops the processing upon identifying the existence of a given risk type. To illustrate, if the method 440 concludes that object weight risk type does not exist, the processing at step 103 next moves to the method 550 (which always concludes with moving to the next evaluation in the hierarchy, the method 660). To continue the illustrative example, the method 660 then determines joint load risk type exists, and such an embodiment then moves to step 104 and outputs an indication that joint load risk type exists. Such an embodiment would not implement the method 770 because the method 660 determined that joint load risk type exists.

According to an embodiment, processing the generated posture at step 103 using the hierarchical decision tree to determine ergonomic risk of the posture comprises evaluating existence of multiple risk types of the posture in a hierarchical order of the multiple risk types. An embodiment of the method 100 utilizes the hierarchical workflow 330, described hereinbelow in relation to FIG. 3, at step 103 to determine ergonomic risk of a posture. Further, another example embodiment stops the processing through the hierarchical decision tree at step 103 upon determining that a given risk type of the multiple risk types exists. In other words, such an embodiment evaluates risk types in a particular order and, upon determining that a risk type exists, lower risk types, i.e., risk types that have not yet been reached in the decision tree, are not considered. In this way, such an embodiment only identifies the highest risk type for a posture. Such an embodiment outputs 104 an indication of the determined ergonomic risk that includes an indication of the given risk type.

According to an embodiment, the hierarchical order of the multiple risk types, in order from first evaluated to last evaluated, at step 103, includes: object weight risk type, hand position risk type, joint load risk type, and body joint angle risk type. Further, it is noted that embodiments are not limited to this order and the aforementioned risk types. Instead, embodiments may evaluate any user desired risk types in any user desired order. The types of risks evaluated and the order with which to evaluate risks at step 103 may be set by a user.

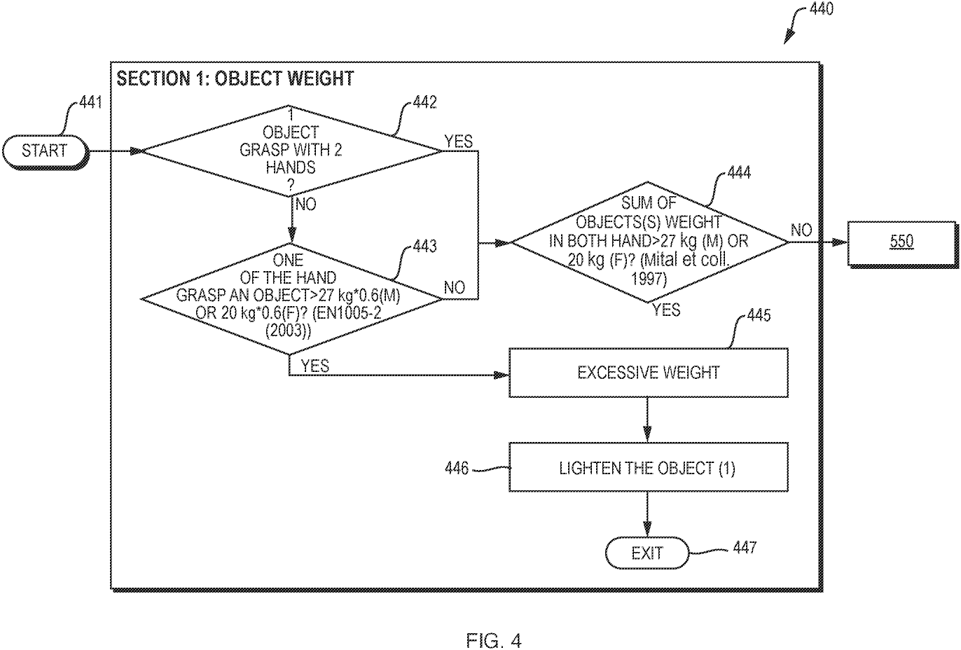

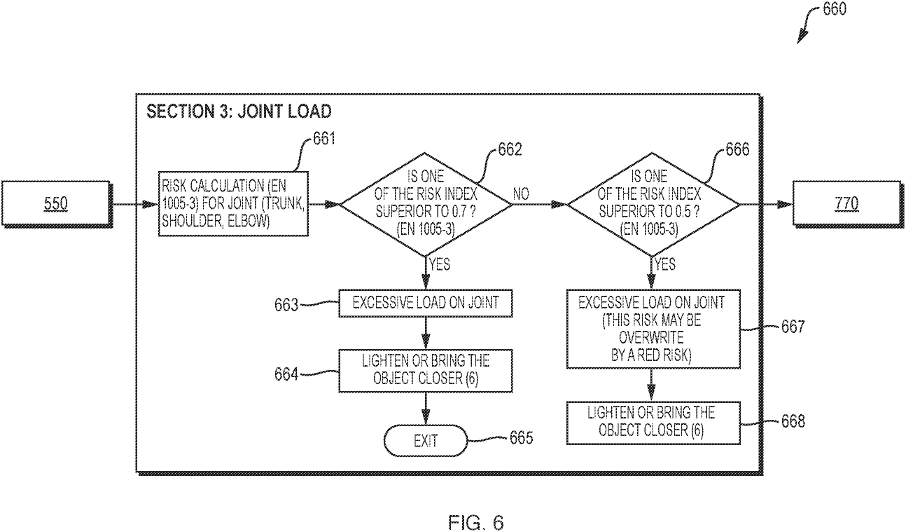

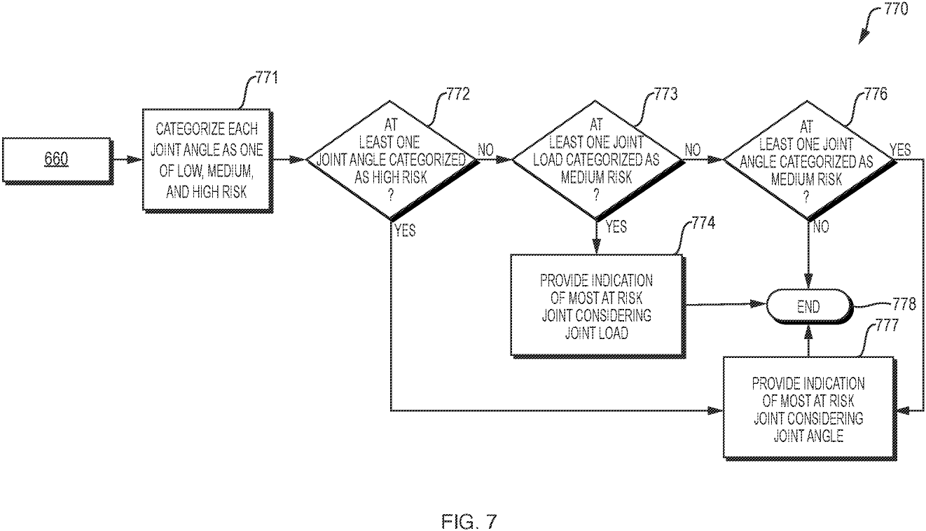

An embodiment of the method 100 evaluates the existence of the object weight risk type at step 103 by comparing weight of an object grasped by the operator performing the task to a threshold. Such an embodiment concludes that the object weight risk type exists if the weight of the object exceeds the threshold. According to an embodiment, a value of the threshold changes based upon the object being grasped with one hand or two hands. Further, an embodiment of the method 100 evaluates the object weight risk type using the method 440 described hereinbelow in relation to FIG. 4. Another embodiment of the method 100 evaluates existence of the joint load risk type at step 103 by first determining at least one of back joint load, shoulder joint load, and elbow joint load of the operator in the generated posture. In turn, the determined at least one of back joint load, shoulder joint load, and elbow joint load are compared to a threshold and the method 100 determines that the joint load risk type exists if the determined at least one of back joint load, shoulder joint load, and elbow joint load exceeds the threshold. An embodiment of the method 100 evaluates the joint load risk type using the method 660 described hereinbelow in relation to FIG. 6. Further, in an embodiment, evaluating existence of the joint angle risk type at step 103 includes comparing at least one of shoulder angle, trunk angle, neck angle, wrist angle, and forearm angle of the operator in the generated posture to respective thresholds (i.e., each body part angle is compared to a threshold that is specific to that body part type). Such an embodiment determines the joint angle risk type exists if at least one of the shoulder angle, trunk angle, neck angle, wrist angle, and forearm angle exceed its respective threshold. An embodiment of the method 100 evaluates the joint angle risk type using the method 770 described hereinbelow in relation to FIG. 7.

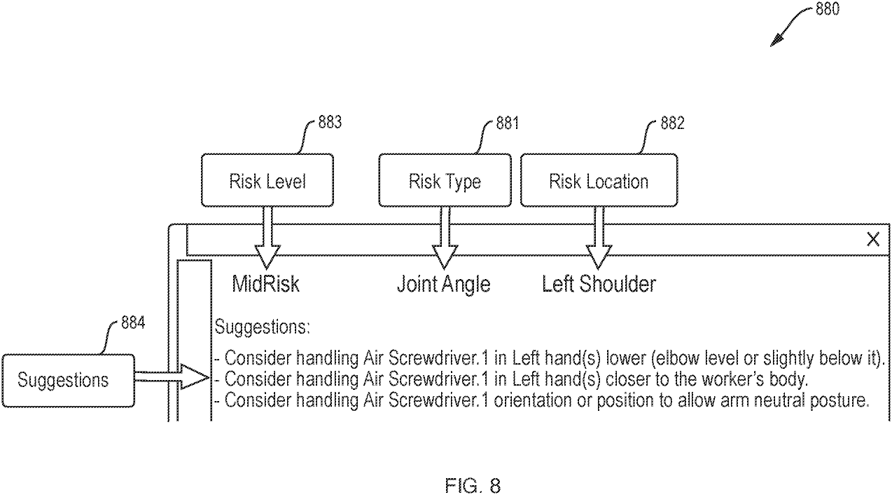

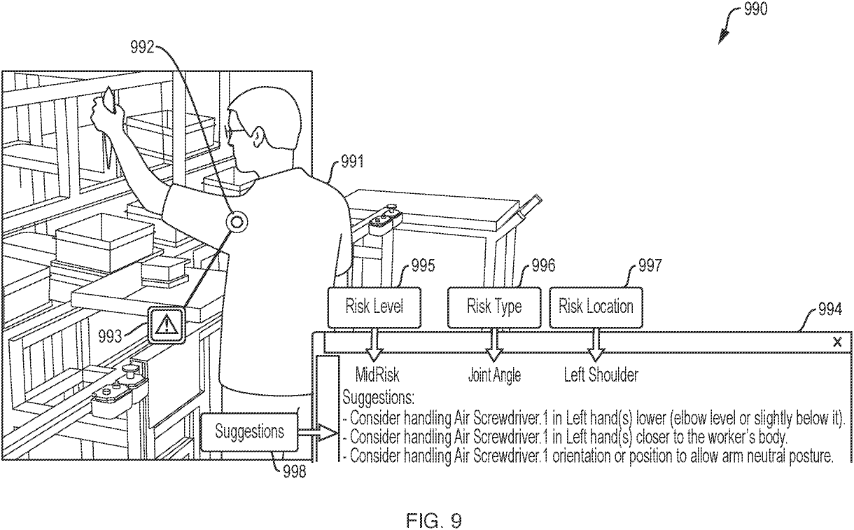

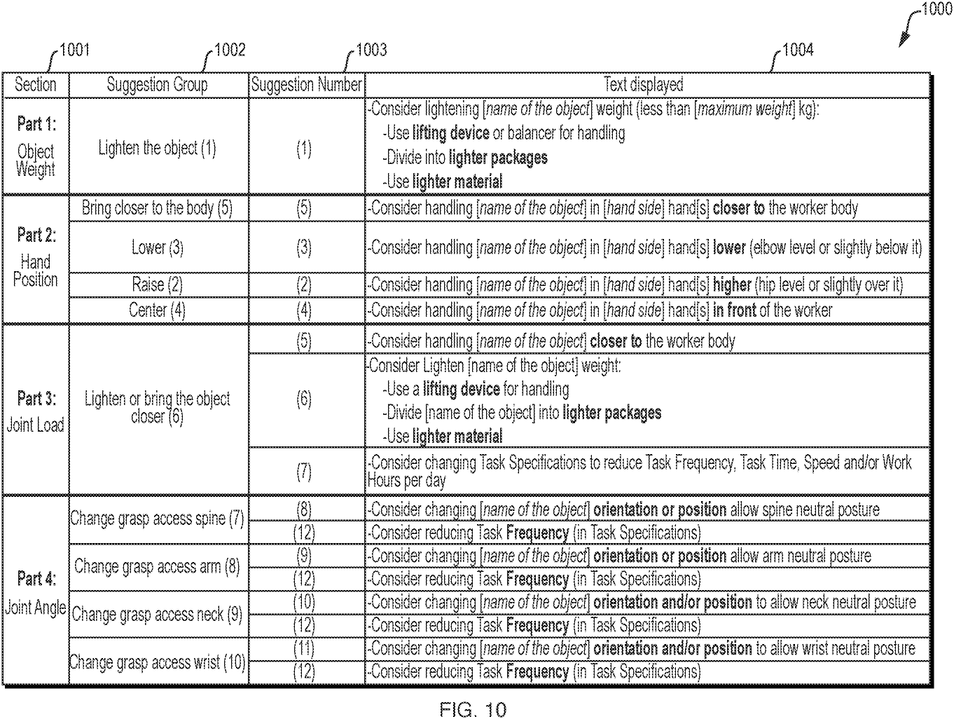

According to an embodiment, the indication of the determined ergonomic risk output at step 104 includes at least one of: a risk type, a risk location, a risk level, and a suggestion to lower risk. Embodiments of the method 100 may provide output at step 104 such as the interfaces 880 and 990, described hereinbelow in relation to FIGS. 8 and 9, respectively. In an example embodiment where the indication of the determined ergonomic risk includes a suggestion, the suggestion may be determined by searching a mapping, such as the table 1000 described hereinbelow in relation to FIG. 10. This mapping, indicates relationships between risk types, risk locations, and suggestions. In such an embodiment, the determined suggestion is mapped to a given risk type and a given risk location of the determined ergonomic risk.

Embodiments of the method 100 may also implement or enable implementation of the suggestion in the certain real-world environment. In this way, embodiments can cause real-world change that improves real-world user ergonomics. For example, if an embodiment determines that joint angle risk type is unacceptable, such an embodiment may suggest bringing the object closer to the worker body and the position of the object may be moved accordingly in the real-world.

### Integration

Embodiments, e.g., Ergo4All™, and the decision tree used therein, can be integrated into existing design software applications, such as Ergonomic Workplace Design (EWD) [4]. Typically, design software applications use process planning data as a starting point. This process planning data contains information related to the product assembly at the workstation, including data regarding the environment and process, e.g., assembling a device, being analyzed. Using EWD, a user can generate worker tasks in 3D, by first indicating a worker action in the form of a sentence that specifies which hand should grasp which object. FIG. 2 illustrates such an example graphical user interface 220 that may be employed by users to input process planning data, e.g., at step 101 of the method 100. FIG. 2 illustrates a task definition, with name 221 “Screwing Task”, where the user has indicated that the right hand 222a is screwing 223 a bolt 224 with an air screw driver 225 having a weight 226 of 0.9 kg. Further, the user has indicated that the left hand 222b is holding 227 an assembly 228 that weighs 229 1 kg in 230 the hand 222b.

From the process data, e.g., as indicated in the interface 220, embodiments use an existing posturing engine, such as the Smart Posturing Engine™, to automatically generate a posture for a manikin, e.g., a digital human model. One such embodiment generates a posture for each of four manikins where each manikin has a different anthropometry, e.g., 5 percentile female, 50 percentile male, 50 percentile female, and 95 percentile male, of the stature of the American population [14, 15]). From these postures, embodiments analyze the combination of posture and force application, indicate the potential risks, and provide suggestions on how to help lower the risks. An example embodiment assesses risk for each posture and outputs an indication of the assessment for each posture.

### Decision Tree Structure

Embodiments employ a hierarchical decision tree processing structure to analyze a posture. FIG. 3 illustrates an example decision tree structure 330 that may be employed by embodiments. The decision tree structure 330 is divided into four sections, 331, 332, 333, and 334, that assess different risk types.

Section 1 (331) assesses object weight. For instance, an embodiment assesses object weight 331 by comparing the weight of an object held by a user to a standard, e.g., EN1005-2 [16] which is based on the revised NIOSH lifting equation, and the work from Mital and his colleagues [17], to ensure the object is not too heavy. Section 2 (332) assesses the hand 335 position relative to the pelvis 336 using a standard, e.g., ISO 14738 [18]. The objective of the hand position assessment 332 is to determine if the hand 335 is too high, too low, too far, etc., in relation to the pelvis 336. Section 3 (333) evaluates the joint loads, e.g., load on shoulder joint 337, using a 3D static biomechanical model. Once the load is determined, the MSD risk score is assessed by comparing the determined load to EN 1005-3 [19]. Section 4 (334) evaluates the body joint angles, e.g., the angle 338 between the upper arm and torso, using EN 1005-4 [20], ISO 11226 [21] and ISO 11228-3 [22].

Within the four sections 331-334 of the tree workflow 330, the risk is prioritized and then presented to a user instantaneously. The objective, according to an embodiment, is to present the worst and most critical risks first before any lower impact risks. For instance, if an object weighs 40 kg, there is no need to proceed to a joint load analysis since the object is simply too heavy for the task to be deemed safe and should be the first risk to be presented and addressed.

### Object Weight

The first element checked by the decision tree, e.g., 330, according to an embodiment is the object weight. According to an embodiment, the acceptable object weight limit is 27 kg for adult males and 20 kg for women [17]. If only one object is grasped with one hand, then the weight limit is 60% of the weight limit of an object grasped with two hands [16]. Further, it is noted that the foregoing standard is but one example, and embodiments may use different standards, e.g., set by users.

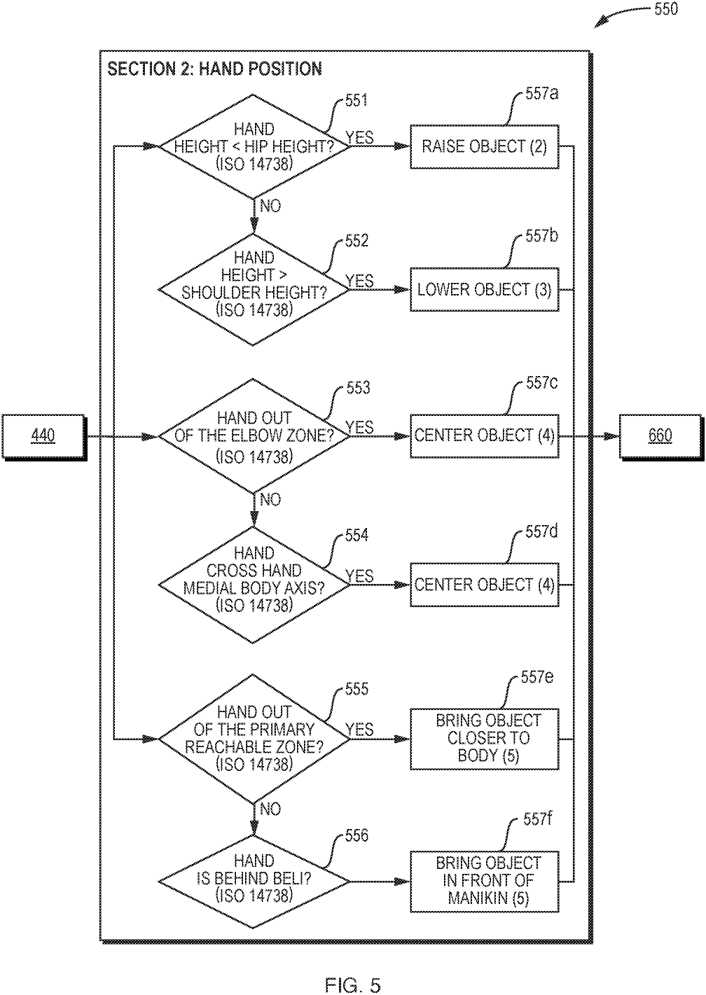

FIG. 4 illustrates a method 440 for analyzing object weight risk according to an embodiment. The method 440 begins 441 and, at step 442, determines if one object is grasped with both hands or if each hand is grasping a separate object. If step 442 determines that a single object is grasped by one hand (no at step 442), the method 440 moves to step 443. At step 443, the method 440 determines if a hand grasps an object that exceeds a threshold (0.6 times 27 kg for males or 0.6 times 20 kg for females) and, if yes, the method 440 moves to step 445. At step 445, the method 440 concludes that the object’s weight is excessive. If the analysis at step 443 determines that the weight of an object in a single hand is not excessive, the method 440 moves to step 444. Likewise, the method 440 can move to step 444 if the analysis at step 442 determines that a single object is grasped with two hands. Regardless of the path to step 444, at step 444, the method 440 determines if the sum of object weight in both hands exceeds a threshold, e.g., 27 kg for males and 20 kg for females. If the object weight is higher than the limit, the method 440 moves to step 445. At step 445 the excessive object weight is deemed to be a high risk and the method 440 moves to step 446 and provides output that includes a suggestion “Lighten the object” and then ends 447. If, however, at step 444 it is determined that the object weight is below the limit, the hierarchical analysis continues by moving to the method 550 of FIG. 5.

### Hand Position

As described above, if the method 440 determines that the object weight meets safety and/or user set standards, the hierarchical processing, e.g., decision tree 330, moves to the method 550 which evaluates hand position relative to the pelvis. In an embodiment, the method 550, follows ISO 14738 [18]. At step 551, the method 550 checks if the is hand too low (below hip height). If step 551 determines the hand is too low (yes at step 551), the method 550 determines output “Raise object” at 557a. If the hand is not too low (no at step 551), the method 550 moves to step 552 and checks if the hand is too high (above shoulder height). If the hand is too high (yes at step 552), the method 550 determines the output “Lower object” at 557b. If step 552 determines the hand is not too high (no at step 552), the method 550 determines there is no hand height issue.

Regardless of the hand height evaluation (steps 551 and 552), the method 550 also checks if a hand is too far to the right/left at steps 553 and 554. Step 553 checks if the hand is outside the elbow zone (e.g., as defined by the ISO14738 standard whereby the “elbow zone” is a function of shoulder width and elbow length). If step 553 determines the hand is outside the elbow zone (yes at step 553), the method 550 determines the output “Center object” at 557c. When the processing at step 553 determines the hand is not outside the elbow zone (no at step 553), the analysis moves to step 554 which examines if a hand is across the median body axis. If a hand is across the medial body axis (yes at step 554) the method 550 determines the output “Center object” at 557d. If a hand is not across the medial body axis (no at step 554) the method 550 determines that no hand is too far to the right or left.

To continue, regardless of the other hand position analyses, the method 550 checks if a hand is too far (step 555) or close (step 556) to the body. At step 555, the method 550 checks if the hand is outside the primary reachable zone (e.g., as defined by the ISO14738 standard whereby the reachable zone is defined as elbow length plus 190 mm) and, if so (yes at step 555), the method 550 determines output “Bring object closer to body” at 557e. If the hand is not too far (no at step 555), the method 550 moves to step 556 and checks if the hand is behind the manikin belly. If the hand is too close (yes at step 556), the method 550 determines the output “Bring object in front of manikin” at 557f. If step 556 determines the hand is not too close, the method 550 determines there is no issue regarding distance between the hand and the body.

Regardless of the analysis at steps 551-556, the method 550 moves to method 660 (FIG. 6) because no risk is associated with the evaluation of the method 550, i.e., no risk is known based on the hand position itself, but the hand position does lead to suggestions, e.g., 557a-f. The method 550 determines suggestions 557a-f if there are issues regarding hand position and determines no suggestions if there are no issues identified by the analyses at steps 551-556. Regardless of suggestions being determined or not, the hierarchical analysis 103, 330 continues to FIG. 6.

### Joint Load

The third section of the hierarchical decision tree analysis (the method 660 of FIG. 6) checks the internal load of the back (trunk), shoulder, and elbow joints. According to an embodiment, the load is the internal static moment in the different planes at the joint location. The method 660 calculates the load using the object weight and manikin joint location in three-dimensional space. The determined loads for the back (trunk), shoulder, and elbow joints are modulated at step 661 using multiplying factors, such as those described in EN 1005-3 [19], while accounting for the task frequency, duration over the work shift, action duration, and velocity. In an embodiment, the modulation at step 661 determines a risk multiplier mᵣ that is, in turn, evaluated at steps 662 and 666. The first step of determining the risk multiplier is determining the EN 1005-3 “reduce capacity” load. The reduce capacity load is the result of the maximal force allowed for a joint following EN1005-3, multiplied by three factors: the velocity multiplier, the frequency multiplier, and the duration multiplier. The reduce capacity load is given by the following equation:

$${F}_{B r} = {F}_{B} \times {m}_{v} \times {m}_{f} \times {m}_{d}$$

Where F_{B} is the maximal force allowed, mᵥ is the velocity multiplier, m_{ƒ} is the frequency multiplier, and m_{d} is the duration multiplier. The multiplier values go from 0 to 1. The slower, less frequent, and less long the joint is solicitated, the higher those multipliers will be (closer to one). Thus, increasing the reduced capacity load value. In turn, the risk multiplier is determined by dividing the joint load (i.e., measured force) by the reduced capacity load according to the following equation:

$${m}_{r} = \frac{{F}_{R}}{{F}_{B r}}$$

Where mᵣ is the risk multiplier, F_{R} is the measured force, and F_{Br} is the reduced capacity force. The result of the modulation 661 is a score above 0 for each joint (back, shoulder, elbow) that represents the percentage of actual joint load compared to the maximum acceptable load. In the method 660, a score higher than 0.7 is considered high risk, a score between 0.5 and 0.7 is considered medium risk, and a score below 0.5 is considered low. Given these ranges and classifications, as described below, the method 660 analyzes the scores determined at step 661.

Step 662 checks if a joint has a score (i.e., risk multiplier mᵣ) higher than 0.7 and, if so (yes at step 662), the joint load is classified as excessive with a high risk level at step 663 and the method 660 determines the suggestion “Lighten or bring the object closer” at step 664 and ends the analysis at 665. If no score is greater than 0.7 (no at step 662), the method 660 moves to step 666. Step 666 checks if a score is greater than 0.5 and, if so (yes at step 666), the joint load is classified as excessive with a medium risk level at step 667 and the method 660 determines the suggestion “Lighten or bring the object closer” at step 668 and, then, continues the hierarchical analysis by moving to method 770 of FIG. 7. This continuation to FIG. 7 is done to determine if there is a high risk level associated with the joint angle. This allows embodiments to ensure that the highest risk is presented to a user. For instance, if a medium risk is identified by the method 660 at step 667, the analysis of FIG. 7 may identify a high risk level for the joint angle and the high joint angle risk would be flagged to a user. Moreover, if medium joint load risk is determined by the method 660 and a medium joint angle risk is found by the method 770, the medium joint load risk is presented to a user. Similarly, if the analysis at step 666 determines that no score is above 0.5, the joint load risk is considered low, and the hierarchical analysis continues to method 770 of FIG. 7.

### Joint Angle

The last section of the hierarchical analysis, e.g., 334 of FIG. 3, checks the angles of joints, e.g., shoulder, back, neck, and wrist. An embodiment follows European and International standards [9-12]. The method 770 of FIG. 7 illustrates an example method 770 that, at step 771 categorizes the joint angle for each joint, based on task frequency, into one of three categories: (1) acceptable, (2) acceptable under condition, and (3) unacceptable. These categories are the equivalent of the three risk levels, low, medium, and high. According to an embodiment, acceptable is associated with a low risk level, acceptable under condition is associated with a medium risk level, and unacceptable is associated with a high risk level. After the categorization of step 771, step 772 determines if there is at least one high risk joint. If there is at least one high risk joint (yes at step 772), the method 770 moves to step 777 and, if there is not (no at step 772) the method 770 moves to step 773. At step 777, the method 770 provides an indication of the most at risk joint, before ending 778.

Returning to step 773, it is determined if there is at least one joint load categorized as medium risk. If there is at least joint load categorized as medium risk (yes at step 773), the method 770 moves to 774 and provides indication of most at the risk joint considering joint load and ends 778. If there is not at least one medium risk joint load (no at step 773), the method 770 moves to step 776. Step 776 determines if there is at least one joint angle categorized as medium risk. If at least one joint angle categorized as medium risk (yes at step 776), the method 770 moves to 777 and provides an indication of the most at risk joint, before ending 778. If there is not at least one joint angle categorized as medium risk (no at step 776), this means that all joint angles are acceptable and the posture is low risk because the previous checks (object weight 440 and joint load 660) were acceptable and hierarchical analysis ends at step 778.

An embodiment only displays one risk for each posture. As such, if a posture has several unacceptable joint angles, a decision is made on which risk to display for a specific posture. This prioritization can be performed at steps 774 and 777 to determine the most at risk joint before providing the indication of the most at risk joint. According to an embodiment, the order of risk to display is the following: (1) unacceptable risks to shoulder, trunk and neck, and (2) acceptable risks under condition to shoulder, trunk, wrist, forearm and neck.

### Output

Embodiments provide an indication of the determined, e.g., by the methods 100, 440, 660, or 770, risk. In an embodiment, the output is in the form of an interface 880 depicted in FIG. 8. The output interface 880 includes an indication of the risk type 881, e.g., object too heavy, joint load, joint angle. Further, the interface 880 includes an indication of the risk location 882, e.g., which joint is at risk: shoulder, back, wrist, neck, elbow, and the risk level 883, e.g., high = red, medium = yellow, low = green. In addition, the interface 880 includes suggestions 884 to improve workstation design. Further details regarding determining the suggestions 884 are described hereinbelow in relation to FIG. 10.

An embodiment, in order to provide easy to use ergonomic guidance, only provides one risk (level, type, and location) at a time for a given work situation. Such an embodiment is designed to efficiently and effectively flag the worst risk for the simulated worker. Meanwhile, there can be several suggestions to improve workstation design and lower the associated risks.

FIG. 9 shows another example output screen 990 that may be provided by embodiments. The output screen 990 illustrates a posture of a manikin 991 generated and automatically analyzed using embodiments described herein. The output 990 includes a dot indicator 992 displayed at the manikin shoulder with a warning sign 993. In an embodiment, the dot indicator 992 can be color or shade coded, to indicate risk level. For example, the dot 992 can be red or yellow to indicate high or medium risk, respectively. The dot 992 by relative displayed location on the manikin body indicates that there is risk on the left shoulder. Further, the screen 990 includes the panel 994 that includes details about the risk assessment (risk level 995, risk type 996, risk location 997, and suggestions 998).

### Suggestions

FIG. 10 is a table 1000 indicating a mapping that is used by embodiments to output suggestions. In particular, the table 1000 is organized into columns including section 1001 (risk type), suggestion group 1002, suggestion number 1003, and output text 1004. The table 1000, thus provides a mapping between the details of the risk, e.g., type 1001 and particular issue 1002, as determined by the analyses (methods 100, 440, 550, 660, and 770) and the suggestion 1004 to provide as output. The suggestion text 1004 is formatted so as to include placeholders, e.g., “[name of object]”, that are completed automatically using word merging techniques based on the process planning data and worker task description defined by the user, e.g., as received at step 101 in method 100 of FIG. 1.

In an embodiment, the suggestions 1004 are derived from the “Ergonomic Checkpoints” book from ILO [23]. When a risk is found by the decision tree analyses (e.g., methods 100, 440, 550, 660, 770), at least one suggestion group 1002 is displayed. Each suggestion group 1002 contains at least one suggestion. Suggestions are numbered from 1 to 12 and are different from each other. Further, it is noted that in embodiments, no risk can be found, and a suggestion can be provided. Amongst other examples, this may occur when no object weight, joint load, or joint angle risk is found, but hand position does not meet a criteria. In such an example, an indication that the posture is low risk is provided along with a suggestion regarding the hand position.

Embodiments provide a new method for assessing ergonomic risk that implements a decision tree that provides ergonomic guidance to manufacturing engineers while designing workstations in 3D. Combined with posturing technology, such as the Smart Posturing Engine™ technology, which generates a posture automatically in a 3D environment, embodiments analyze the potential risk of developing MSD by workers. Embodiments provide guidance to users on changes to environments, e.g., workstations, that will lower the ergonomic risks.

Advantageously, embodiments provide simple ergonomic guidance to engineers that do not have training in ergonomics. The decision tree processing used in embodiments provides a rapid assessment tool for users designing and evaluating environments, such as real-world workstations.

Embodiments and the hierarchical decision tree processing described herein can utilize existing standards, which are based on ergonomics methods published in scientific journals, to evaluate the posture and force combination of a manikin performing an action in a 3D environment. Advantageously, embodiments organize and order the processing to provide coherent information to users while designing or otherwise evaluating work environments. Embodiments can rely on a number of different standards because no unique ergonomics standard considers all aspects, e.g., object weigh, hand location, joint load and angle, needed to analyze the posture in relation to the environment.

Embodiments help manufacturing engineers to design safe workplaces. Embodiments can be linked to posturing software, such as the Smart Posturing Engine™ developed by the applicant, that automatically generates manikin postures in 3D. This combination (embodiments and posturing software) provides very quick assessments of workstations, e.g., 3D simulated work workstations or workstations as they exist in the real-world.

Unlike existing methods, embodiments advantageously analyze joint load and joint angle when assessing postures. Further, embodiments provide suggestions, e.g., 1004, that are targeted to the identified risk. Moreover, these suggestions are actionable improvements that can be implemented in the real-world to mitigate MSD risks. In addition, embodiments can stop processing when existence of a risk is identified in the hierarchical analysis. This maximizes computational efficiency. Moreover, because the processing is hierarchical, embodiments identify the highest risk elements which more efficiently lead to solutions without any or limited risks. Further, user interfaces, e.g., 880, indicating one risk are more easily interpreted by users.

### Computer Support

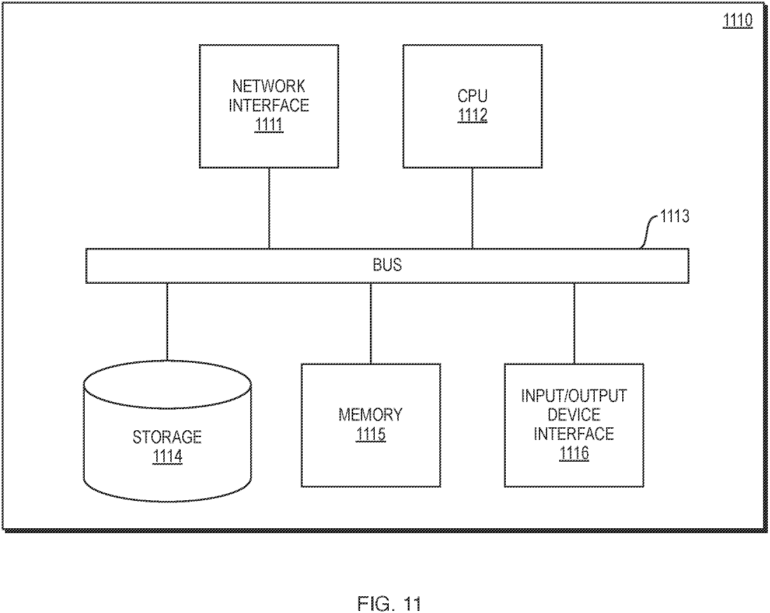

FIG. 11 is a simplified block diagram of a computer-based system 1110 that may be used to assess ergonomic risk according to any variety of the embodiments of the present invention described herein. The system 1110 comprises a bus 1113. The bus 1113 serves as an interconnect between the various components of the system 1110. Connected to the bus 1113 is an input/output device interface 1116 for connecting various input and output devices such as a keyboard, mouse, display, speakers, etc. to the system 1110. A central processing unit (CPU) 1112 is connected to the bus 1113 and provides for the execution of computer instructions. Memory 1115 provides volatile storage for data used for carrying out computer instructions. In particular, memory 1115 and storage 1114 hold computer instructions and data (databases, tables, etc.) for carrying out methods 100, 330, 440, 550, 660, 770 of FIGS. 1, 3, 4, 5, 6, and 7 and supporting corresponding user interfaces 880, 990 described above. Storage 1114 provides non-volatile storage for software instructions, such as an operating system (not shown). The system 1110 also comprises a network interface 1111 for connecting to any variety of networks known in the art, including wide area networks (WANs) and local area networks (LANs).

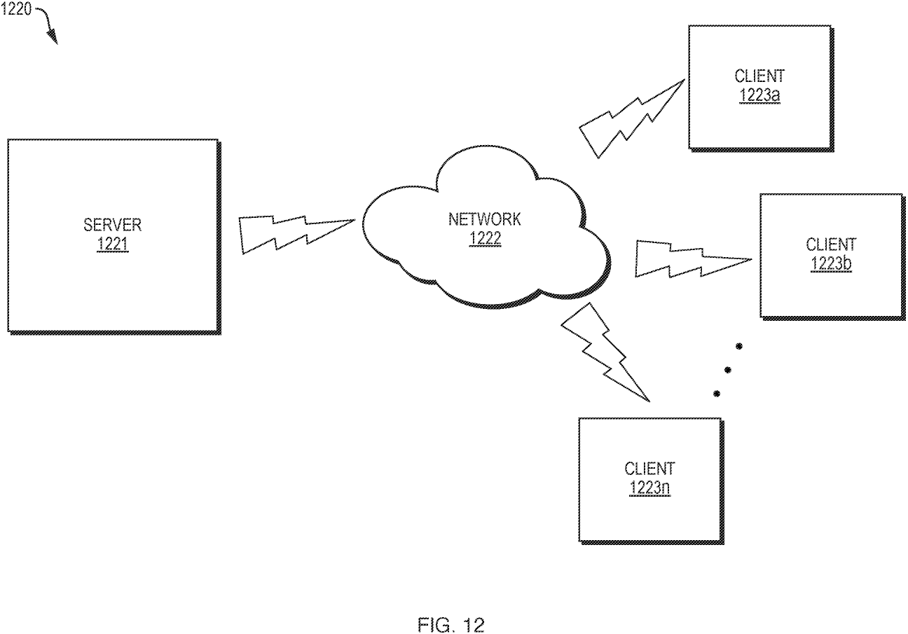

It should be understood that the example embodiments described herein may be implemented in many different ways. In some instances, the various methods and machines described herein may each be implemented by a physical, virtual, or hybrid general purpose computer, such as the computer system 1110, or a computer network environment such as the computer environment 1220, described herein below in relation to FIG. 12. The computer system 1110 may be transformed into the machines that execute the methods (e.g., 100, 330, 440, 550, 660, 770) and techniques described herein, for example, by loading software instructions into either memory 1115 or non-volatile storage 1114 for execution by the CPU 1112. One of ordinary skill in the art should further understand that the system 1110 and its various components may be configured to carry out any embodiments or combination of embodiments of the present invention described herein. Further, the system 1110 may implement the various embodiments described herein utilizing any combination of hardware, software, and firmware modules operatively coupled, internally, or externally, to the system 1110.

FIG. 12 illustrates a computer network environment 1220 in which an embodiment of the present invention may be implemented. In the computer network environment 1220, the server 1221 is linked through the communications network 1222 to the clients 1223a-n. The environment 1220 may be used to allow the clients 1223a-n, alone or in combination with the server 1221, to execute any of the embodiments described herein. For non-limiting example, computer network environment 1220 provides cloud computing embodiments, software as a service (SAAS) embodiments, and the like.

Embodiments or aspects thereof may be implemented in the form of hardware, firmware, or software. If implemented in software, the software may be stored on any non-transient computer readable medium that is configured to enable a processor to load the software or subsets of instructions thereof. The processor then executes the instructions and is configured to operate or cause an apparatus to operate in a manner as described herein.

Further, firmware, software, routines, or instructions may be described herein as performing certain actions and/or functions of the data processors. However, it should be appreciated that such descriptions contained herein are merely for convenience and that such actions in fact result from computing devices, processors, controllers, or other devices executing the firmware, software, routines, instructions, etc.

It should be understood that the flow diagrams, block diagrams, and network diagrams may include more or fewer elements, be arranged differently, or be represented differently. But it further should be understood that certain implementations may dictate the block and network diagrams and the number of block and network diagrams illustrating the execution of the embodiments be implemented in a particular way.

Accordingly, further embodiments may also be implemented in a variety of computer architectures, physical, virtual, cloud computers, and/or some combination thereof, and thus, the data processors described herein are intended for purposes of illustration only and not as a limitation of the embodiments.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

### References

1. Stephens, A., Jones, M.: Workplace methods and use of digital human models. In: Handbook of Digital Human Modeling, pp. 6.1-6.11. Taylor and Francis, USA (2009).

2. Santos Human. https://www.santoshumaninc.com/. Accessed 22 Jan. 2021

3. Blanchonette, P.: Jack human modelling tool: a review. Air Operations Division Defence Science and Technology Organisation (2010).

4. Ergonomics at Dassault Systèmes. https://www.3ds.com/products-services/delmia/disciplines/industrial-engineering/tag/88-1007/. Accessed 22 Jan. 2021.

5. Cort, J.A., Devries, D.: Accuracy of postures predicted using a digital human model during four manual exertion tasks, and implications for ergonomic assessments. IISE Trans. Occup. Ergon. Hum. Factors 7(1), 43-58 (2019).

6. Hanson, L., Högberg, D., Carlson, J.S., Bohlin, R., Brolin, E., Delfs, N., et al.: IMMA-Intelligently moving manikins in automotive applications. In: Third International Summit on Human Simulation (ISHS 2014) (2014).

7. McAtamney, L., Corlett, E.N.: RULA: a survey method for the investigation of work-related upper limb disorders. Appl. Ergon. 24(2), 91-99 (1993).

8. Hignett, S., McAtamney, L.: Rapid entire body assessment (REBA). Appl. Ergon. 31(2), 201-205 (2000).

9. Waters, T.R., Putz-Anderson, V., Garg, A., Fine, L.J.: Revised NIOSH equation for the design and evaluation of manual lifting tasks. Ergonomics 36(7), 749-776 (1993).

10. Chaffin, D.: Engineers with HFE education-Survey results. HFES-ETG News Lett. 3, 2-3 (2005).

11. Lemieux, P.-O., Barré, A., Hagemeister, N., Aissaoui, R.: Degrees of freedom coupling adapted to the upper limb of a digital human model. Int. J. Hum. Factors Model. Simul. 5(4), 314-337 (2017).

12. Lemieux, P., Cauffiez,M., Barré, A., Hagemeister, N., Aissaoui, R.: A visual acuity constraint for digital human modeling. In: 4th Conference proceedings (2016).

13. Zeighami, A., Lemieux, P., Charland, J., Hagemeister, N., Aissaoui, A.: Stepping behavior for stability control of a digital human model. ISB/ASB (2019).

14. Reed, M.P., Raschke, U., Tirumali, R., Parkinson, M.B.: Developing and implementing parametric human body shape models in ergonomics software. In: Proceedings of the 3^{rd} International Digital Human Modeling Conference, Tokyo (2014).

15. Parkinson,M.B., Reed,M.P.: Creating virtual user populations by analysis of anthropometric data. Int. J. Ind. Ergon. 40(1), 106-111 (2010).

16. EN 1005-2. Safety of machinery-Human physical performance. Part 2: Manual handling of machinery and component parts of machinery. Brussels, European Committee for Standardization (2003).

17. Mital, A.: Guide to Manual Materials Handling. CRC Press, Boca Raton (1997).

18. ISO 14738: Sécurité des machines-Prescriptions anthropométriques relatives à la conception des postes de travail sur lesmachines. Geneva: International Organization for Standardization (2003). (in French)

19. EN1005-3. Safety of machinery-Human physical performance. Part 3: Recommended force limits for machinery operation. Brussels, European Committee for Standardization (2003).

20. EN 1005-4. Safety of machinery-Human physical performance. Part 4: Evaluation of Working Postures and Movements in Relation to Machinery. Brussels, European Committee for Standardization (2003).

21. ISO 11226. Ergonomics - Evaluation of static postures. Geneva: International Organization for Standardization (2000).

22. ISO 11228-3. Ergonomics.Manual handling: Part 3: Handling of low loads at high frequency. Geneva: International Organization for Standardization (2010).

23. International Labour Office and International Ergonomics Association: Ergonomic Checkpoints: Practical and Easy-to-Implement Solutions for Improving Safety, Health and Working Conditions. International Labour Organization, Geneva (1996).

24. Quentin Bourret, Pierre-Olivier Lemieux, Julie Charland, Rachid Aissaoui. Grasp Planning Of Unknown Object For Digital Human Model. 13th International Conference on Applied Human Factors and Ergonomics.

What is claimed is:

1. A computer-implemented method of assessing ergonomic risk, the method comprising, by a processor:
receiving process planning data for an operator performing a task;
using the received process planning data, generating a posture for the operator to perform the task in a certain real-world environment;
processing the generated posture using a hierarchical decision tree to determine ergonomic risk of the posture in the certain real-world environment; and
outputting an indication of the determined ergonomic risk, said outputting being to at least a user.

2. The method of claim 1 wherein the process planning data includes at least one of:
physical characteristics of a workstation in the certain real-world environment at which the task is performed;
physical characteristics of the operator; and
characteristics of the task.

3. The method of claim 1 wherein processing the generated posture using the hierarchical decision tree to determine ergonomic risk of the posture comprises:
evaluating existence of multiple risk types of the posture in a hierarchical order of the multiple risk types;
upon determining a given risk type of the multiple risk types exists, stopping the evaluating; and
outputting the indication of the determined ergonomic risk, wherein the indication of the determined ergonomic risk includes an indication of the given risk type.

4. The method of claim 3 wherein the hierarchical order of the multiple risk types, in order from first evaluated to last evaluated includes: object weight risk type, hand position risk type, joint load risk type, and body joint angle risk type.

5. The method of claim 4 wherein evaluating existence of the object weight risk type comprises:
comparing weight of an object grasped by the operator performing the task to a threshold, wherein a value of the threshold changes based upon the object being grasped with one hand or two hands; and
determining the object weight risk type exists if the weight of the object exceeds the threshold.

6. The method of claim 4 wherein evaluating existence of the joint load risk type comprises:
determining at least one of back joint load, shoulder joint load, and elbow joint load of the operator in the generated posture;
comparing the determined at least one of back joint load, shoulder joint load, and elbow joint load to a threshold; and
determining the joint load risk type exists if the determined at least one of back joint load, shoulder joint load, and elbow joint load exceeds the threshold.

7. The method of claim 4 wherein evaluating existence of the joint angle risk type comprises:
comparing at least one of shoulder angle, trunk angle, neck angle, wrist angle, and forearm angle of the operator in the generated posture to respective thresholds; and
determining the joint angle risk type exists if at least one of the shoulder angle, trunk angle, neck angle, wrist angle, and forearm angle exceed a respective threshold.

8. The method of claim 1 wherein the indication of the determined ergonomic risk includes at least one of:
a risk type;
a risk location;
a risk level; and
a suggestion to lower risk.

9. The method of claim 8 wherein the indication of the determined ergonomic risk includes the suggestion and the method further comprises:
determining the suggestion by searching a mapping between risk types, risk locations, and suggestions, wherein the determined suggestion is mapped to a given risk type and a given risk location of the determined ergonomic risk.

10. The method of claim 9 further comprising:
implementing the suggestion in the certain real-world environment.

11. The method of claim 1 wherein receiving the process planning data comprises:
receiving a measurement from a sensor in the certain real-world environment in which the task is performed.

12. A system for assessing ergonomic risk, the system comprising:
a processor; and
a memory with computer code instructions stored thereon, the processor and the memory, with the computer code instructions, being configured to cause the system to:
receive process planning data for an operator performing a task;
using the received process planning data, generate a posture for the operator to perform the task in a certain real-world environment;
process the generated posture using a hierarchical decision tree to determine ergonomic risk of the posture in the certain real-world environment; and
output an indication of the determined ergonomic risk, said outputting being to at least a user.

13. The system of claim 12 wherein, in processing the generated posture using the hierarchical decision tree to determine ergonomic risk of the posture, the processor and the memory, with the computer code instructions, are further configured to cause the system to:
evaluate existence of multiple risk types of the posture in a hierarchical order of the multiple risk types;
upon determining a given risk type of the multiple risk types exists, stop the evaluating; and
output the indication of the determined ergonomic risk, wherein the indication of the determined ergonomic risk includes an indication of the given risk type.

14. The system of claim 13 wherein the hierarchical order of the multiple risk types, in order from first evaluated to last evaluated includes: object weight risk type, hand position risk type, joint load risk type, and body joint angle risk type.

15. The system of claim 14 wherein, in evaluating existence of the object weight risk type, the processor and the memory, with the computer code instructions, are further configured to cause the system to:
compare weight of an object grasped by the operator performing the task to a threshold, wherein a value of the threshold changes based upon the object being grasped with one hand or two hands; and
determine the object weight risk type exists if the weight of the object exceeds the threshold.

16. The system of claim 14 wherein, in evaluating existence of the joint load risk type, the processor and the memory, with the computer code instructions, are further configured to cause the system to:
determine at least one of back joint load, shoulder joint load, and elbow joint load of the operator in the generated posture;
compare the determined at least one of back joint load, shoulder joint load, and elbow joint load to a threshold; and
determine the joint load risk type exists if the determined at least one of back joint load, shoulder joint load, and elbow joint load exceeds the threshold.

17. The system of claim 14 wherein, in evaluating existence of the joint angle risk type, the processor and the memory, with the computer code instructions, are further configured to cause the system to:
compare at least one of shoulder angle, trunk angle, neck angle, wrist angle, and forearm angle of the operator in the generated posture to respective thresholds; and
determine the joint angle risk type exists if at least one of the shoulder angle, trunk angle, neck angle, wrist angle, and forearm angle exceed a respective threshold.

18. The system of claim 12 wherein the indication of the determined ergonomic risk includes at least one of:
a risk type;
a risk location;
a risk level; and
a suggestion to lower risk.

19. The system of claim 18 wherein the indication of the determined ergonomic risk includes the suggestion and, the processor and the memory, with the computer code instructions, are further configured to cause the system to:
determine the suggestion by searching a mapping between risk types, risk locations, and suggestions, wherein the determined suggestion is mapped to a given risk type and a given risk location of the determined ergonomic risk.

20. A non-transitory computer program product for assessing ergonomic risk, the computer program product executed by a server in communication across a network with one or more client and comprising:
a computer readable medium, the computer readable medium comprising program instructions which, when executed by a processor, causes the processor to:
receive process planning data for an operator performing a task;
using the received process planning data, generate a posture for the operator to perform the task in a certain real-world environment;
process the generated posture using a hierarchical decision tree to determine ergonomic risk of the posture in the certain real-world environment; and
output an indication of the determined ergonomic risk, said outputting being to at least a user.
